(19) United States
(12) Patent Application Publication
(10) Pub. No.: US 2014/0039572 A1
(43) Pub. Date: Feb. 6, 2014

(54) SYSTEM AND METHOD FOR TREATING DEPRESSION AND EPILEPSY

(71) Applicant: BOSTON SCIENTIFIC NEUROMODULATION CORPORATION, Valencia, CA (US)

(72) Inventor: Kerry Bradley, Glendale, CA (US)

(73) Assignee: BOSTON SCIENTIFIC NEUROMODULATION CORPORATION, Valencia, CA (US)

(21) Appl. No.: 13/958,218

(22) Filed: Aug. 2, 2013

Related U.S. Application Data
(60) Provisional application No. 61/679,271, filed on Aug. 3, 2012.

(51) Int. Cl. A61N 1/36

(52) U.S. Cl. CPC A61N 1/36064; A61N 1/36096
USPC 607/45

(57) ABSTRACT

A method for treating a patient suffering from epilepsy and/or depression includes applying electrical stimulation energy to a first cervical segment and/or a second cervical segment of a spinal cord of the patient, thereby treating the epilepsy and/or depression. The electrical stimulation energy may be epidurally applied to the first cervical segment and/or the second cervical segment of the spinal cord of the patient by an electrode implanted within an epidural space of the patient adjacent to the first or second cervical segment of the spinal cord. The electrode may be implanted adjacent to the first cervical vertebra and/or the second cervical vertebra of the patient. Applying the electrical stimulation energy may include applying electrical activation energy to activate afferent pathways that feed collateral nerve fibers into a trigeminocervical complex of the patient. The applied electrical stimulation energy may have a frequency in the range of 40-50 Hz.

### RELATED APPLICATION DATA

The present application claims the benefit under 35 U.S.C. §119 to U.S. provisional patent application Ser. No. 61/679,271, filed Aug. 3, 2012. The foregoing application is hereby incorporated by reference into the present application in its entirety.

### FIELD OF THE INVENTION

The present invention relates to tissue stimulation systems, and more particularly, to spinal cord stimulation systems.

### BACKGROUND OF THE INVENTION

Implantable neurostimulation systems have proven therapeutic in a wide variety of diseases and disorders. For example, Spinal Cord Stimulation (SCS) techniques, which directly stimulate the spinal cord tissue of the patient, have long been accepted as a therapeutic modality for the treatment of chronic neuropathic pain syndromes, and the application of spinal cord stimulation has expanded to include additional applications, such as angina pectoralis, peripheral vascular disease, and incontinence, among others. Spinal cord stimulation is also a promising option for patients suffering from motor disorders, such as Parkinson's Disease, Dystonia and essential tremor.

An implantable SCS system typically includes one or more electrode-carrying stimulation leads, which are implanted at a stimulation site in proximity to the spinal cord tissue of the patient, and a neurostimulator implanted remotely from the stimulation site, but coupled either directly to the stimulation lead(s) or indirectly to the stimulation lead(s) via a lead extension. The neurostimulation system may further include a handheld patient programmer to remotely instruct the neurostimulator to generate electrical stimulation pulses in accordance with selected stimulation parameters. The handheld programmer may, itself, be programmed by a technician attending the patient, for example, by using a Clinician's Programmer (CP), which typically includes a general purpose computer, such as a laptop, with a programming software package installed thereon.

Thus, programmed electrical pulses can be delivered from the neurostimulator to the stimulation lead(s) to stimulate or activate a volume of the spinal cord tissue. In particular, electrical stimulation energy conveyed to the electrodes creates an electrical field, which, when strong enough, depolarizes (or “stimulates”) the neural fibers within the spinal cord beyond a threshold level, thereby inducing the firing of action potentials (APs) that propagate along the neural fibers to provide the desired efficacious therapy to the patient. Additionally, the nerve fibers of the spinal cord may be electrically modulated. Electrical modulation of nerve fibers can alter the activity of the neurons by affecting their resting state (e.g. held in a state of relative depolarization or hyperpolarization) or their activity (e.g. their ability to transmit action potentials). Electrical modulation allows for altering the performance of neurons without necessarily causing them to create APs.

Different types of stimulation have been shown to be somewhat effective for treating depression and/or epilepsy. For example, vagus nerve stimulation and deep brain stimulation have been shown to have moderate efficacy for treating depression and/or epilepsy. However, vagus nerve stimulation and deep brain stimulation are highly invasive procedures that are typically not tested on a trial basis. Recently, trigeminal nerve stimulation has been shown to have efficacy in treating epilepsy and depression. Trigeminal nerve stimulation is non-invasive and can be trialed using cutaneous patches coupled to an electrical stimulator.

Thus, there remains a need for alternative systems and methods for treating depression and/or epilepsy that are less invasive than vagus nerve stimulation or deep brain stimulation, and that may be used as an alternative to cutaneous trigeminal nerve stimulation.

### SUMMARY OF THE INVENTION

In accordance with the present inventions, a method for treating a patient suffering from at least one of epilepsy and depression is provided. The method includes applying electrical stimulation energy (e.g., at a frequency in the range of 40-50 Hz) to at least one of a first cervical segment and a second cervical segment of a spinal cord of the patient, thereby treating the at least one of epilepsy and depression. The method may be for treating epilepsy, depression, or both epilepsy and depression. The electrical stimulation energy may be applied at the first cervical segment, the second cervical segment, or both the first cervical segment and the second cervical segment of the spinal cord of the patient. The electrical stimulation energy may be epidurally applied to the at least one of the first cervical segment and the second cervical segment of the spinal cord of the patient. The electrical stimulation energy may be applied by at least one electrode implanted within an epidural space of the patient adjacent to the first or second cervical segment of the spinal cord. The electrical stimulation energy may be applied by at least one electrode implanted adjacent to at least one of a first cervical vertebra or a second cervical vertebra of the patient. Applying the electrical stimulation energy may include applying electrical activation energy to activate afferent pathways that feed collateral nerve fibers into a trigeminocervical complex of the patient.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present invention are obtained, a more particular description of the present invention briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Spinal Cord Stimulation (SCS) system constructed in accordance with one embodiment of the present inventions;

FIG. 2 is a plan view of the SCS system of FIG. 1 in use within a patient;

FIG. 3 is a plan view of an implantable pulse generator (IPG) and three percutaneous stimulation leads used in the SCS system of FIG. 1;

FIG. 4 is a plan view of an implantable pulse generator (IPG) and a surgical paddle lead used in the SCS system of FIG. 1;

FIG. 5 is a peripheral view of the spinal cord and spinal nerves;

FIG. 6 is a cross-sectional view of the spinal cord; and

FIG. 7 is a plan view of the trigeminocervical complex.

### DETAILED DESCRIPTION OF THE EMBODIMENTS

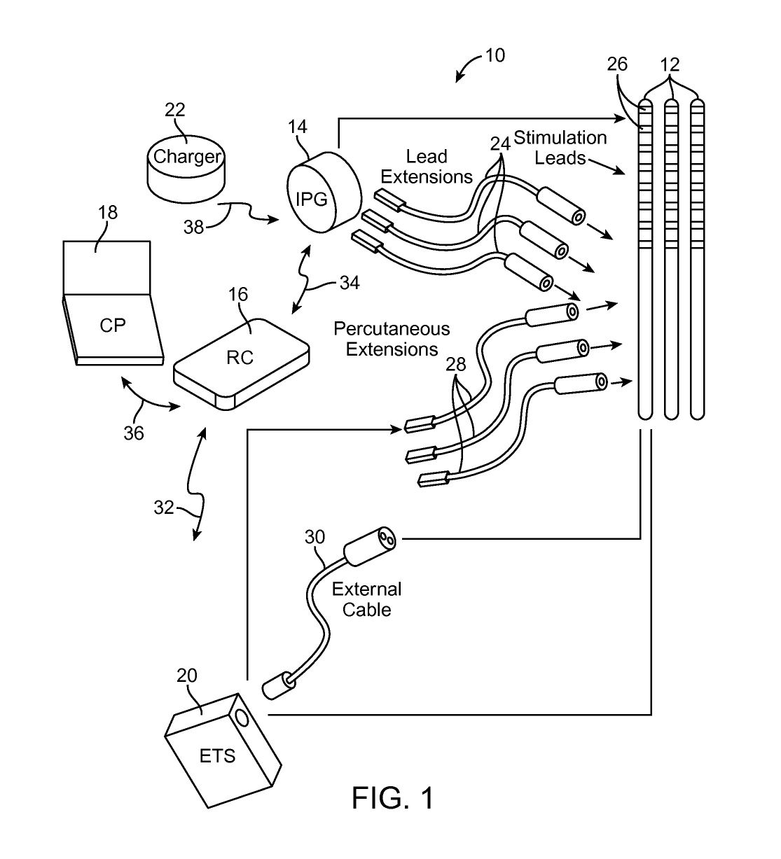

Turning first to FIG. 1, an exemplary SCS system 10 generally comprises a plurality of stimulation leads 12 (in this case, three), an implantable pulse generator (IPG) 14 (or alternatively RF receiver-stimulator), an external remote control RC 16, a Clinician's Programmer (CP) 18, an External Trial Stimulator (ETS) 20, and an external charger 22.

The IPG 14 is physically connected via one or more lead extensions 24 to the stimulation leads 12, which carry a plurality of electrodes 26 arranged in an array. The stimulation leads 12 are illustrated as percutaneous leads in FIG. 1, although as will be described in further detail below, a surgical paddle lead can be used in place of the percutaneous leads. As will also be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and an external cable 30 to the neurostimulation leads 12. The ETS 20, which has similar pulse generation circuitry to the IPG 14, also delivers electrical stimulation energy in the form of a pulse electrical waveform to the electrode array 26 in accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Thus, any functions described herein with respect to the IPG 14 can likewise be performed with respect to the ETS 20.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be described herein. Details of exemplary embodiments of these devices are disclosed in U.S. Pat. No. 6,895,280, which is expressly incorporated herein by reference.

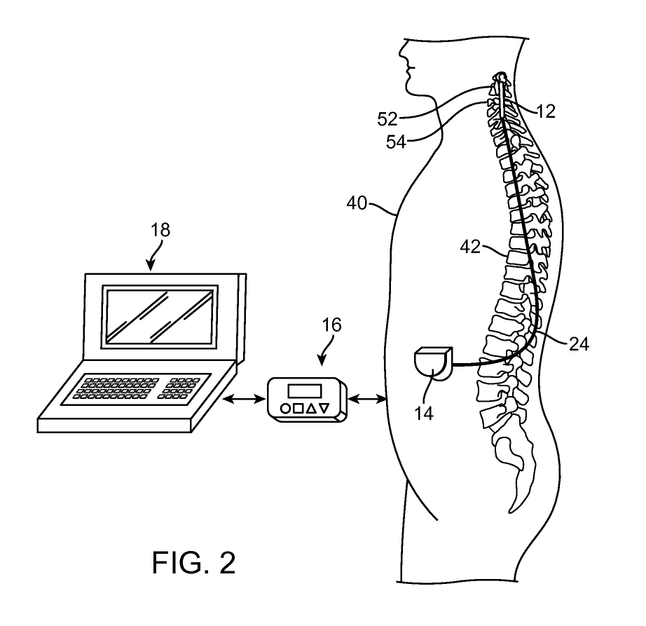

As shown in FIG. 2, the stimulation leads 12 are implanted within the spinal column 42 of a patient 40. In accordance with the present invention, the stimulation leads 12 are implanted within the upper cervical region of the spinal column 42. For example, the leads 12 may be implanted adjacent to the first and/or second cervical segments of the spinal cord, which are generally surrounded by the first cervical vertebra 52 and the second cervical vertebra 54.

The preferred placement of the stimulation leads 12 is adjacent, i.e., resting near, the spinal cord area to be stimulated. Due to the lack of space near the location where the stimulation leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the stimulation leads 12. As there shown, the CP 18 communicates with the IPG 14 via the RC 16.

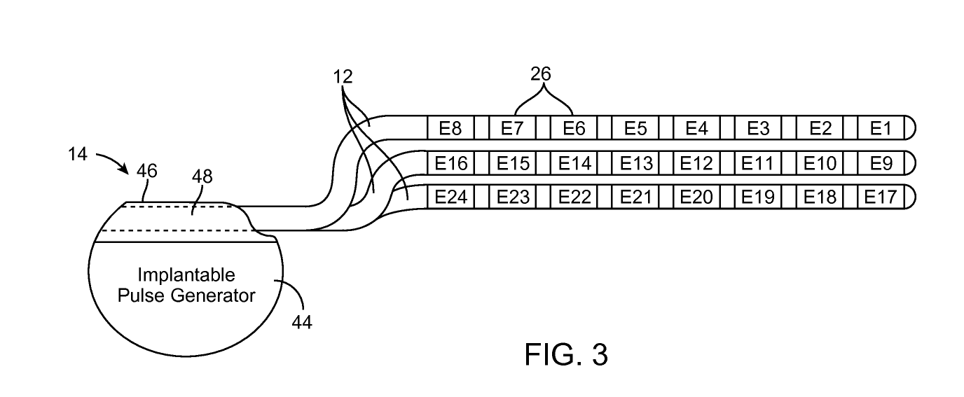

Referring now to FIG. 3, the external features of the stimulation leads 12 and the IPG 14 will be briefly described. Each of the stimulation leads 12 has eight electrodes 26 (respectively labeled E1-E8, E9-E16, and E17-E24). The actual number and shape of leads and electrodes will, of course, vary according to the intended application. Further details describing the construction and method of manufacturing percutaneous stimulation leads are disclosed in U.S. Pat. No. 8,019,439, entitled “Lead Assembly and Method of Making Same,” and U.S. Pat. No. 7,650,184, entitled “Cylindrical Multi-Contact Electrode Lead for Neural Stimulation and Method of Making Same,” the disclosures of which are expressly incorporated herein by reference.

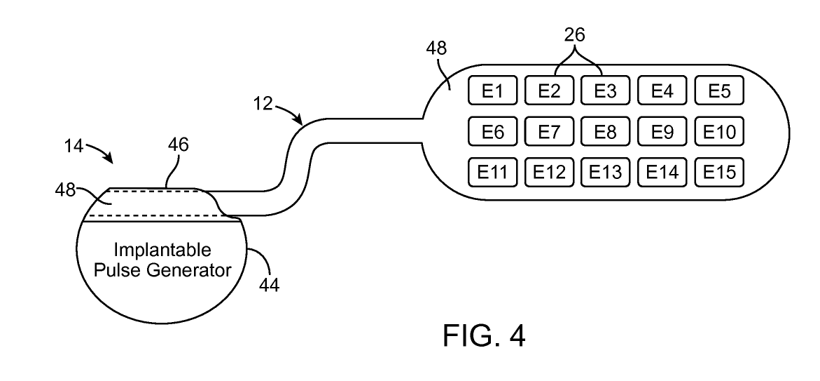

Alternatively, as illustrated in FIG. 4, the stimulation lead 12 takes the form of a surgical paddle lead on which electrodes 26 are arranged in a two-dimensional array in three columns (respectively labeled E1-E5, E6-E10, and E11-E15) along the axis of the stimulation lead 12. In the illustrated embodiment, five rows of electrodes 26 are provided, although any number of rows of electrodes can be used. Each row of the electrodes 26 is arranged in a line transversely to the axis of the lead 12. The actual number of leads and electrodes will, of course, vary according to the intended application. Further details regarding the construction and method of manufacture of surgical paddle leads are disclosed in U.S. Patent Application Publication No. 2007/0150036, entitled “Stimulator Leads and Methods for Lead Fabrication,” the disclosure of which is expressly incorporated herein by reference.

In each of the embodiments illustrated in FIGS. 3 and 4, the IPG 14 comprises an outer case 44 for housing the electronic and other components (described in further detail below). The outer case 44 is composed of an electrically conductive, biocompatible material, such as titanium, and forms a hermetically sealed compartment wherein the internal electronics are protected from the body tissue and fluids. In some cases, the outer case 44 may serve as an electrode. The IPG 14 further comprises a connector 46 to which the proximal ends of the stimulation leads 12 mate in a manner that electrically couples the electrodes 26 to the internal electronics (described in further detail below) within the outer case 44. To this end, the connector 46 includes one or more ports 48 (three ports for three percutaneous leads or one port for the surgical paddle lead) for receiving the proximal end(s) of the stimulation lead(s) 12. In the case where the lead extensions 24 are used, the port(s) 48 may instead receive the proximal ends of such lead extensions 24.

The IPG 14 includes pulse generation circuitry that provides electrical conditioning and stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse width (measured in microseconds), pulse rate (measured in pulses per second), and cycling rate (measured as the ‘stimulation on’ duration X and ‘stimulation off’ duration Y).

Electrical stimulation will occur between two (or more) activated electrodes, one of which may be the IPG case 44. Simulation energy may be transmitted to the tissue in a monopolar or multipolar (e.g., bipolar, tripolar, etc.) fashion. Monopolar stimulation occurs when a selected one of the lead electrodes 26 is activated along with the case 44 of the IPG 14, so that stimulation energy is transmitted between the selected electrode 26 and the case 44. Bipolar stimulation occurs when two of the lead electrodes 26 are activated as anode and cathode, so that stimulation energy is transmitted between the selected electrodes 26. For example, an electrode on one lead 12 may be activated as an anode at the same time that an electrode on the same lead or another lead 12 is activated as a cathode. Tripolar stimulation occurs when three of the lead electrodes 26 are activated, two as anodes and the remaining one as a cathode, or two as cathodes and the remaining one as an anode. For example, two electrodes on one lead 12 may be activated as anodes at the same time that an electrode on another lead 12 is activated as a cathode.

The stimulation energy may be delivered between electrodes as monophasic electrical energy or multiphasic electrical energy. Monophasic electrical energy includes a series of pulses that are either all positive (anodic) or all negative (cathodic). Multiphasic electrical energy includes a series of pulses that alternate between positive and negative. For example, multiphasic electrical energy may include a series of biphasic pulses, with each biphasic pulse including a cathodic (negative) stimulation pulse and an anodic (positive) recharge pulse that is generated after the stimulation pulse to prevent direct current charge transfer through the tissue, thereby avoiding electrode degradation and cell trauma. That is, charge is conveyed through the electrode-tissue interface via current at an electrode during a stimulation period (the length of the stimulation pulse), and then pulled back off the electrode-tissue interface via an oppositely polarized current at the same electrode during a recharge period (the length of the recharge pulse).

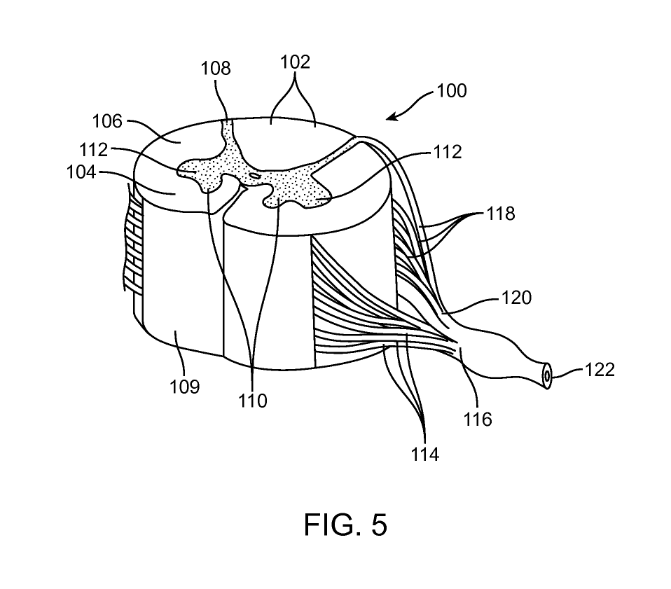

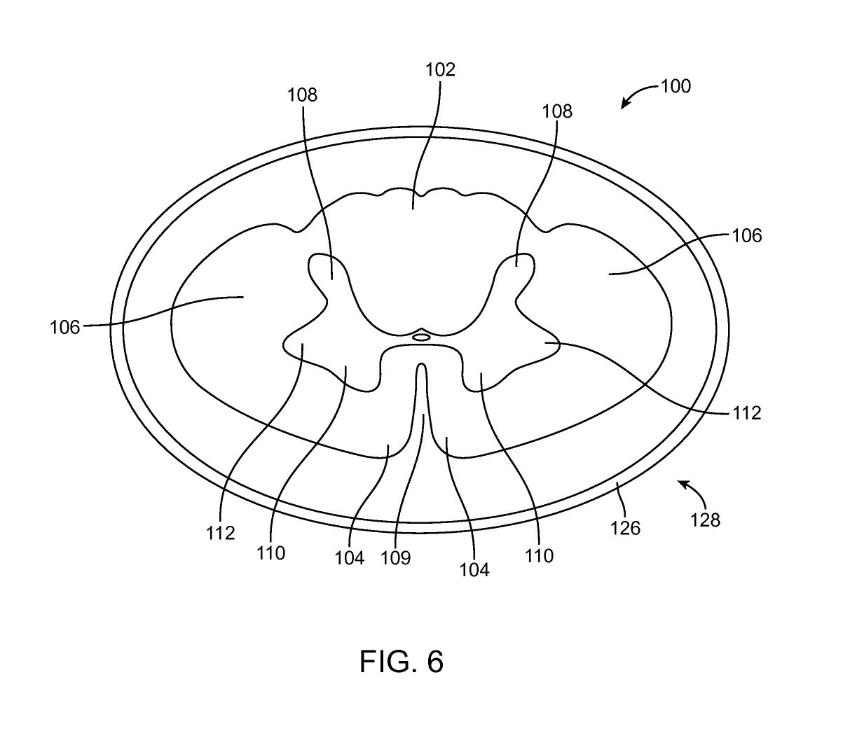

Referring now to FIGS. 5 and 6, the portions of the spinal cord 100 that are relevant to the present inventions will be described. The spinal cord 100 is divided into three functional columns: the dorsal column 102, the ventral column 104, and the lateral columns 106. Similarly, the butterfly-shaped gray matter of the spinal cord 100 is divided into the dorsal horn 108, the ventral horn 110, and the lateral horn 112. A ventral median fissure 109 divides the spinal cord 100 into two lateral halves. The spinal cord 100 is enclosed by a dura mater 126, with an epidural space 128 surrounding the dura mater 126.

A group of motor nerve rootlets (ventral root (VR) nerve fibers) 114 branch off of the ventral horn 110 and combine to form the ventral root 116. Similarly, a group of sensory nerve rootlets (dorsal root (DR) nerve fibers) 118 branch off of the dorsal horn 108 and combine to form the dorsal root 120. The dorsal root 120 and the ventral root 116 combine to form the spinal nerve 122, which innervates peripheral regions (e.g., arms, legs, etc.) of the patient's body. A number of spinal nerves branch off the spinal cord. In each patient, there are eight cervical spinal nerves designated C1-C8, twelve thoracic spinal nerves designated T1-T12, five lumbar spinal nerves designated L1-L5, and five sacral spinal nerves designated S1-S5.

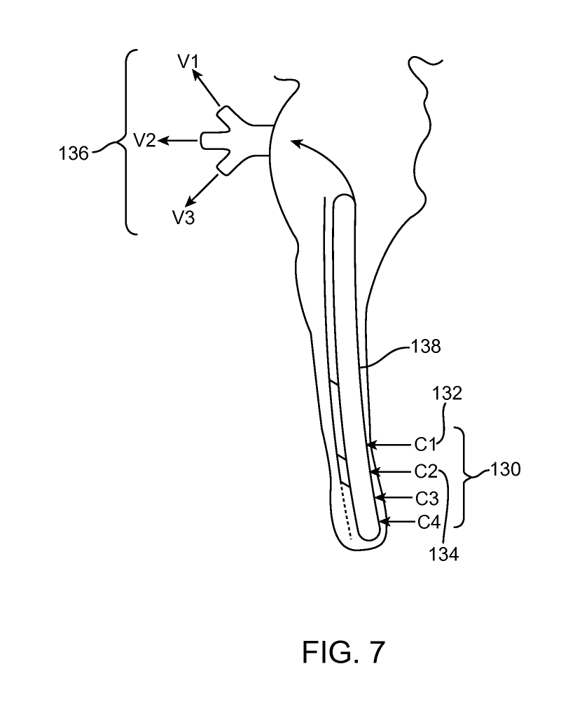

Of particular importance to the present invention are the first and second cervical segments of the spinal cord 100, which are generally surrounded by the first cervical vertebra 52 and the second cervical vertebra 54, shown in FIG. 2. As shown in FIG. 7, the pathway from the upper cervical region 130 of the spinal cord 100 (which includes the first cervical segment (C1) 132 and the second cervical segment (C2) 134 of the spinal cord) to the trigeminal nerves 136 of the head and face is through the trigeminocervical nucleus 138, which extends through the brainstem and into the cervical cord 130. Thus, as an alternative to trigeminal nerve stimulation, stimulation of afferent pathways that feed collateral nerve fibers into the trigeminocervical complex may be useful in treating epilepsy and/or depression.

In a method for treating a patient suffering from epilepsy and/or depression, the SCS system 10 is used to apply stimulation to the C1 132 and/or C2 134 segments of the spinal cord 100. To this end, the stimulation lead(s) 12 are preferably implanted within the patient adjacent to the spinal cord 100 with the distal ends of the stimulation lead(s) 12 positioned within the epidural space 128 surrounding the C1 132 and/or C2 134 segments of the spinal cord 100. Since the C1 132 and C2 134 segments of the spinal cord 100 are generally surrounded by the first cervical vertebra 52 and the second cervical vertebra 54, the stimulation lead(s) 12 may be implanted within the patient so that at least of the electrodes 26 is positioned adjacent to the first cervical vertebra 52 and/or the second cervical vertebra 54.

Thus, electrical stimulation energy is epidurally applied by the stimulation lead(s) 12 to the C1 segment 132 of the spinal cord 100, the C2 segment 134 of the spinal cord 100, or both the C1 132 and C2 134 segments of the spinal cord 100, thereby treating epilepsy, depression, or both epilepsy and depression. The electrical stimulation energy is applied in a manner that causes activation of afferent pathways that feed collateral nerve fibers into the trigeminocervical complex of the patient. The applied electrical stimulation energy may have a frequency in the range of 40-50 Hertz, an amplitude in the range of 2-3 mA, and a pulse width of about 200 μs. It should be well understood that these stimulation parameters are merely exemplary and that the stimulation parameters of the electrical stimulation energy may be different from those recited herein.

During application of the stimulation energy to the C1 132 and/or C2 134 segments of the spinal cord 100, it may be desirable to avoid inadvertently creating side-effects, e.g., in the form of uncomfortable muscle contractions and pain resulting from the inadvertent stimulation of the DR and/or VR nerve fibers. The electrodes 26 may be arranged relative to the VR nerve fiber grouping 114 and/or DR nerve fiber grouping 116 in any one of a variety of manners. For example, the stimulation lead(s) 12 may be positioned adjacent to the dorsal column 102 or ventral column 104 of the spinal cord 100, thereby easily avoiding inadvertent stimulation of the DR and/or VR nerve fibers.

Because the stimulation leads(s) are located within the cervical region of the spinal cord, wherein the sensitive VR nerve fibers 114 and DR nerve fibers 118 extend straight out from the spinal cord 100, more spatial isolation is provided between these nerve fibers and the lateral column 106 of the spinal cord 100. As such, the distal tip(s) of the stimulation lead(s) 12 may be easily located between the VR nerve fiber grouping 114 and/or DR nerve fiber grouping 116 rostral and caudal to the positioned stimulation leads.

Further, inadvertent stimulation of the DR and/or VR nerve fibers may be avoided by increasing the activation threshold of neural structures (e.g., the VR nerve fibers 114 and/or DR nerve fibers 118) relative to the activation threshold of the lateral column 106 of the spinal cord 100. Different techniques can be used to increase the activation thresholds of these neural structures relative to the activation threshold of the lateral column 106 of the spinal cord 100. Such techniques are discussed in greater detail in U.S. Provisional Patent Application Ser. No. 61/569,214, which is expressly incorporated herein by reference.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A method for treating a patient suffering from at least one of epilepsy and depression, the method comprising:
applying electrical stimulation energy to at least one of a first cervical segment and a second cervical segment of a spinal cord of the patient, thereby treating the at least one of epilepsy and depression.

2. The method of claim 1, wherein the least one of epilepsy and depression comprises epilepsy.

3. The method of claim 1, wherein the at least one of epilepsy and depression comprises depression.

4. The method of claim 1, wherein the electrical stimulation energy is epidurally applied to the at least one of the first cervical segment and the second cervical segment of the spinal cord of the patient.

5. The method of claim 1, wherein the electrical stimulation energy is applied to the first cervical segment of the spinal cord of the patient.

6. The method of claim 1, wherein the electrical stimulation energy is applied to the second cervical segment of the spinal cord of the patient.

7. The method of claim 1, wherein the electrical stimulation energy is applied to the first cervical segment and the second cervical segment of the spinal cord of the patient.

8. The method of claim 1, wherein the electrical stimulation energy is applied by at least one electrode implanted within an epidural space of the patient adjacent to the first or second cervical segment of the spinal cord.

9. The method of claim 1, wherein the electrical stimulation energy is applied by at least one electrode implanted adjacent to at least one of a first cervical vertebra and a second cervical vertebra of the patient.

10. The method of claim 1, wherein applying the electrical stimulation energy comprises applying electrical activation energy to activate afferent pathways that feed collateral nerve fibers into a trigeminocervical complex of the patient.

11. The method of claim 1, wherein the applied electrical stimulation energy has a frequency in the range of 40-50 Hz.
